(19) United States
(12) Patent Application Publication
(10) Pub. No.: US 2003/0215472 A1
(43) Pub. Date: Nov. 20, 2003

(54) Methods and compositions employing a dialkyl amide

(76) Inventors: Craig A. Bonda, Winfield, IL (US); David C. Steinberg, Plainsboro, NJ (US); Gary A. Neudahl, Cary, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/334,584

(22) Filed: Dec. 31, 2002

Related U.S. Application Data
(60) Provisional application No. 60/381,339, filed on May 16, 2002.

(51) Int. Cl. A61K 7/00; A61K 9/00; A61K 31/16

(52) U.S. Cl. 424/401; 514/625; 424/47

(57) ABSTRACT

A deodorant composition including a dialkyl amide in a dermatologically acceptable carrier, a method of preparing a cosmetic product intended for human topical application including the deodorant composition, a method for controlling malodor associated with human perspiration including the step of applying to the skin the deodorant composition, and a combination for use as a deodorant on human skin including the deodorant composition and a tube container, are disclosed herein.

### CROSS REFERENCE To RELATED APPLICATION

The benefit under 35 U.S.C. §119(e) of U.S. Provisional Patent Application Serial No. 60/381,339 filed May 16, 2002, is hereby claimed.

### BACKGROUND

1. Field of the Invention

The invention generally relates to preparations for use on the skin. More particularly, the invention relates to inhibition of body odor by a deodorant ingredient, a deodorant composition containing the ingredient, and a method of suppressing malodor formation by applying such a composition.

2. Brief Description of Related Technology

Deodorant compositions are well known for use in controlling malodors associated with human perspiration. These malodors develop from human perspiration primarily as the result of microbial (e.g., axillary bacteria) interaction with sweat gland secretions which then produces pungent fatty compounds. For example, such secretions include proteinaceous materials from eccrine and apocrine glands, such as amino acids and sialomycin, and fatty materials such as triglycerides from sebaceous glands. Two primary types of products that suppress body odor, deodorants and antiperspirants, are in wide commercial use today in toiletries and cosmetics. The former group can be subdivided into two subgroups: products containing an active material which suppresses or inhibits the growth of microorganisms present on the skin and thereby prevents their action on sweat to produce odoriferous substances, and products containing odor absorbers such as activated charcoal, cyclodextrins, and zeolites. The latter group of antiperspirants includes products which contain active materials that suppress or inhibit sweating and thereby remove or reduce a component that contributes to production of odiferous substances. The active materials can also be combined to produce an antiperspirant/deodorant composition. For various reasons, such as aesthetic preference, sensitivity to certain astringent antiperspirant salts, and the like, some consumers who wish to use an odor-controlling product prefer a product that includes an active ingredient that suppresses or inhibits the growth of microorganisms present on the skin.

Deodorant formulations are typically applied topically to the underarm and surrounding areas of the skin, and in addition to being effective at controlling or masking perspiration malodors a deodorant formulation can be formulated to provide various aesthetic characteristics, such as clarity, ease of application, a cool and refreshing feel on application, lack of powdery residue, and a dry feel.

It is well known that many deodorant products contain ingredients that suppress or inhibit the growth of bacteria (including axillary bacteria); however, not all bacteristatic or bactericidal compounds will necessarily produce an effective deodorant product. For example, although many strains and species of bacteria exist, only a very few specific strains colonize the surface of the skin and are responsible for producing malodor. Bacteria associated with axillary odor include Corynebacterium xerosis and Micrococcus luteus. Bacteristatic and bactericidal compounds exhibit a wide range of potencies against any particular bacterial strain. For example, a particular compound may be highly potent against one strain but ineffective against another. Good antibacterial deodorant compounds must therefore be effective against the specific strains of bacteria that cause malodor.

Moreover, an effective antibacterial deodorant ingredient must be safe, i.e., non-toxic to the body and non-irritating to the skin with often daily application of an amount effective to inhibit the growth of bacteria.

In addition, an effective antibacterial deodorant ingredient preferably maintains its activity for the desired length of time in the location of application (e.g., the axilla). Thus, an effective antibacterial deodorant ingredient preferably is physically, chemically, and biochemically compatible with the product formulation and the environmental conditions of the location of application, and it preferably is sufficiently adherent to the skin so it is not easily rubbed off.

### SUMMARY

One aspect of the disclosure provides a composition including a dialkyl amide in a dermatologically acceptable carrier.

Another aspect of the disclosure provides a method of preparing a cosmetic product intended for human topical application including the step of including in the product a composition including a dialkyl amide in a dermatologically acceptable carrier.

Still another aspect of the disclosure provides a method for controlling a condition selected from acne and malodor associated with human perspiration, including the step of applying to the skin a composition including a dialkyl amide in a dermatologically acceptable carrier.

Yet another aspect of the disclosure provides a combination for use as a deodorant on human skin including a deodorant composition including a dialkyl amide in a dermatologically acceptable carrier and a tube container for the deodorant composition.

Further aspects and advantages may become apparent to those skilled in the art from a review of the following detailed description, taken in conjunction with the appended claims. While the invention is susceptible of embodiments in various forms, described hereinafter are specific embodiments with the understanding that the disclosure is illustrative, and is not intended to limit the invention to the specific embodiments described herein.

### DETAILED DESCRIPTION

The invention includes a deodorant composition containing a dialkyl amide and method of suppressing body odor by the topical application of a deodorant composition containing a dialkyl amide in a dermatologically acceptable carrier. The invention also includes an anti-acne composition containing a dialkyl amide and a method of suppressing acne by the topical application of an anti-acne composition containing a dialkyl amide in a dermatologically acceptable carrier.

The dialkyl amide preferably is derived from a C₈-C₁₈ monocarboxylic acid, and more preferably is a N,N,-dimethyl amide. The compounds include straight-chain and branched-chain species, and saturated and unsaturated species, including species with multiple unsaturation sites. Preferred are straight-chain aliphatic acids, either saturated or unsaturated, of 8 to 18 carbon atom length, preferably 10 to 14 carbon atom length. Examples include capric (decanoic), undecanoic, lauric, tridecanoic, myristic, myristoleic, pentadecanoic, palmitic, palmitoleic, hexadecanoic, margaric, oleic, linoleic, linolenic, and octadecanoic acids. Examples of amides include N,N-dimethyldecanamide (dimethyl capramide), N,N-dimethylundecanamide, N,N-dimethyllauramide, N,N-dimethyltridecanamide, N,N-diemethylmyristamide, N,N-dimethylmyristoleylamide, N,N-dimethylpentadecanamide, and N,N-dimethylpalmitamide. Preferably, the dialkyl amide will suppress or inhibit the growth of axillary bacteria.

Dialkyl amides can be prepared by amidation of an organic acid in liquid or vapor phase, e.g., as disclosed in U.S. Pat. No. 2,667,511 (Jan. 26, 1954), U.S. Pat. No. 3,006,956 (Oct. 31, 1961), and U.S. Pat. No. 3,468,919 (Sep. 23, 1969).

A preferred dialkyl amide is N,N-dimethyldecanamide, which as been found to be non-irritating and non-sensitizing to human skin when used at levels effective for suppressing malodor formation. The dialkyl amide N,N-dimethyldecanamide (CAS Registry No. 14433-76-2) is also effective in killing and suppressing gram-positive bacteria. The dialkyl amide N,N-dimethyldecanamide also has been found to have excellent solubility characteristics. For example, it is soluble in an amount greater than 50 wt. % in propylene glycol and greater than 50 wt. % in cyclomethicone. Thus, for example, a dialkyl amide such as N,N-dimethyldecanamide can be used to supplement or replace triclosan in any deodorant formulation.

One or more dialkyl amides preferably is present in the composition in an amount effective to suppress malodor formation and/or to suppress acne formation. Generally, a dialkyl amide preferably will be present in an amount from 0.01 wt. % to 10 wt. %, more preferably 0.1 wt. % to 2 wt. %. In one deodorant embodiment, a dialkyl amide will be present in an amount at least 0.032%, preferably at least 0.8%. In one anti-acne embodiment, a dialkyl amide will be present in an amount at least 0.16%, preferably at least 0.5%.

The compositions can take any form convenient for application to the skin, e.g., for use in suppressing acne or malodor formation. The dermatologically acceptable carrier can be aqueous or anhydrous. For example, the carrier can include a vehicle selected from the group consisting of water, ethanol, a dihydric alcohol, a polyhydric alcohol, a silicone, a fatty alcohol, a fatty acid, a metallic salt of a fatty acid (e.g., sodium stearate and magnesium stearate), a fatty acid ester, and combinations thereof.

The compositions can be formulated as any one of a solution, lotion, cream, ointment, powder, suspension, stick, gel, and aerosol, for example. Sticks and gels are preferred for deodorants, whereas lotions, creams, ointments, and gels are preferred for anti-acne compositions. For example, the deodorant composition can be formulated as a deodorant stick, optionally including a gellant (e.g., a metallic stearate such as potassium stearate) or other structurant, and a polar alcohol solvent to help solubilize the gellant or other structurant. The deodorant composition can also be formulated as an emulsion gel including a dihydric or polyhydric alcohol, or combination thereof, in a silicone. An example includes an emulsion of polypropylene glycol in cyclomethicone.

Optionally, a deodorant or anti-acne composition including a dialkyl amide that is compatible with surfactant systems can be formulated into cleansing products such as shower gels, body washes, and the like.

If desired, a deodorant composition as described herein can include an antiperspirant compound, such as an antiperspirant salt. In such a formulation, a dialkyl amide as disclosed herein can be incorporated into an antiperspirant formulation with the antiperspirant being employed in an amount effective to reduce perspiration.

The antiperspirant component can be, for example, any of those which contain aluminum, either alone or in combination with other materials such as zirconium. Typical aluminum salts, although not all-inclusive, include: aluminum chlorohydrate, aluminum sesquichlorohydrate, aluminum dichlorohydrate, aluminum chlorohydrex PG and PEG, aluminum sesquichlorohydrex PG and PEG, aluminum dichlorohydrex PG and PEG, aluminum zirconium trichlorohydrate, aluminum zirconium tetrachlorohydrate, aluminum zirconium tetrachlorohydrex PG and PEG, aluminum zirconium pentachlorohydrate, aluminum zirconium octachlorohydrate, aluminum zirconium trichlorohydrex-gly, aluminum zirconium tetrachlorohydrex-gly, aluminum zirconium pentachlorohydrex-gly, aluminum zirconium octachlorohydrex-gly, aluminum zirconium chloride, aluminum zirconium sulfate, potassium aluminum sulfate, sodium aluminumchlorohydroxylacetate, and aluminum bromohydrate.

In general, the active antiperspirant component should be present in the same amounts at which such materials are conventionally employed. For example, a composition according to the disclosure that includes an antiperspirant should contain from 5 wt. % to 30 wt. %, preferably from 10 wt. % to 25 wt. % of an active antiperspirant salt component.

The compositions can further include any other optional component otherwise known or found to be suitable for use in an anti-acne or a deodorant composition that includes an antimicrobial active. Examples include, but are not limited to, surfactants, rheology modifiers, emulsifiers, fillers, skin care actives, and other compounds known and commonly used in the art.

The deodorant composition can further include a germicide selected from the group consisting of triclosan, zinc phenol sulfonate and chlorhexidine digluconate in amounts ranging from 0.01 wt. % to 10 wt. %, preferably from 0.1 wt. % to 5 wt. % of the deodorant composition.

Another aspect of the invention is a method of preparing a cosmetic product intended for human topical application, the method including the step of including in the product a composition including a dialkyl amide as described herein.

Still another aspect of the invention is a method for controlling malodor associated with human perspiration, the method including the step of applying to the skin a deodorant composition as described herein. Preferably, the application step will include applying the deodorant composition to the underarm and optionally areas of the skin surrounding the underarm. The application step can optionally include applying the deodorant composition to one or both feet.

Yet another aspect of the invention is a method for controlling acne, the method including the step of applying to the skin a composition including a dialkyl amide as described herein.

Another aspect of the invention is a combination for use as a deodorant on human skin including a deodorant composition as described herein and a tube container for the deodorant composition. The tube container is constructed to expose a small amount of the product at an open end of the container. Optionally, the containers can include a removable cap over the open end to enclose the deodorant composition when it is stored or not in use. For example, the combination can include a deodorant composition as described herein disposed in a tube container having a base end and dispensing end and a base rotatable relative to the base end of the tube wherein rotation of the base relative to the tube causes the deodorant composition to be displaced along the longitudinal axis of the tube out of the dispensing end.

### EXAMPLES

### Example 1

The objective of this experiment was to determine the antimicrobial activity of the test sample dimethyl capramide (the INCI or cosmetic name for N,N-dimethyldecanamide) by determining the Minimal Inhibitory Concentration and Maximum Lethal Concentration via Zone of Inhibition testing against Micrococcus luteus, Propoinibacterium acnes and Corynebacterium xerosis.

For culture preparation, an overnight Tryptic Soy Agar (TSA) culture was grown with M. luteus ATCC #10240. This culture was washed and diluted with saline to 85% transmittance at 530 nm. An overnight anaerobic culture of P. acnes ATCC#11827 was prepared in Brain Heart Infusion Broth (BHIB). This was washed with saline and diluted to 85% transmittance at 530 nm. An overnight aerobic culture of C. xerosis ATCC#373 was also grown in BHIB, washed with saline and diluted to 85% transmittance at 530 nm. These cultures were used as inocula. Three sets of nine different dilutions of sample were prepared for MIC results using TSA or BHIB as follows: (1) 2.0 grams sample+8.0 mL media=1:5 or 20%; (2) 2.0 mL of #1+8.0 mL media=1:25 or 4%; (3) 2.0 mL of #2+8.0 mL media=1:125 or 0.8%; (4) 2.0 mL of #3+8.0 mL media=1:625 or 0.16%; (5) 2.0 mL of #4+8.0 mL media=1:3125 or 0.032%; (6) 2.0 mL of #5+8.0 mL media=1:15625 or 0.0064%; (7) 2.0 mL of #6+8.0 mL media=1:78125 or 0.00128%; (8) 2.0 mL of #7+8.0 mL media=1:390625 or 0.000256%; and (9) 2.0 mL of #8+8.0 mL media=1:1953125 or 0.0000512%.

The product dilutions were dispensed onto Zone of Inhibition plates. TSA and BHIB plates were prepared by using the base and seed method. Seed consisted of 2.0 mL of inoculum per 20.0 mL of TSA or BHIB used. A single penicylinder placed in the center of the plate when agar was solidified. Each penicylinder was inoculated with 200 μL of the MIC dilutions listed above; this procedure was performed in duplicate. Control plates were performed simultaneously; also using the base and seed method. M. luteus plates were incubated for 48 hours at 30° C. to 35° C. P. acnes plates were incubated for 5-7 days 36° C. to 38° C. anaerobically, C. xerosis plates were incubated aerobically at 36° C. to 38° C. Plates were measured for zones and calculated in millimeters.

For MLC Preparation, swabbing of the zones was performed to calculate MLC results. Each plate that showed marked zone areas was swabbed. These swabs were then placed in TSA or BHIB for incubation. M. luteus tubes were incubated for 48 hours at 30° C. to 35° C. P. acnes tubes were incubated for 5-7 days at 35° C. to 38° C. anaerobically, C. xerosis tubes were incubated aerobically at 36° C. to 38° C. Tubes were inspected visually for growth and indicated below. Results are shown in Tables 1, and 2, below. Numbers indicate the average of the zone sizes on the two plates (in millimeters) as per MIC dilution. MLC results are recorded as positive or negative for growth.

TABLE 1
MIC/Zone of Inhibition

| | Dilution: | | | | | | | | |
|---|---|---|---|---|---|---|---|---|---|
| Bacterium | 1:5 | 1:25 | 1:125 | 1:625 | 1:3125 | 1:15625 | 1:78125 | 1:390625 | 1:1953125 |
| M. luteus | 14.7 | 14.2 | 13.6 | NZ | NZ | NZ | NZ | NZ | NZ |
| P. acnes | 21.2 | 23.8 | 18.0 | 8.1 | NZ | NZ | NZ | NZ | NZ |
| C. xerosis | 20.2 | 13.9 | 17.3 | 8.6 | 7.2 | NZ | NZ | NZ | NZ |

NZ = No Zone

TABLE 2
MLC Results

| | Dilution: | | | | | | | | |
|---|---|---|---|---|---|---|---|---|---|
| Bacterium | 1:5 | 1:25 | 1:125 | 1:625 | 1:3125 | 1:15625 | 1:78125 | 1:390625 | 1:953125 |
| M luteus | (−) | (+) | (+) | NA | NA | NA | NA | NA | NA |
| P.acnes | (−) | (−) | (+) | (+) | NA | NA | NA | NA | NA |
| C. xerosis | (−) | (−) | (−) | (+) | (+) | NA | NA | NA | NA |

NA = Not applicable

The dimethyl capramide composition was effective, giving an MIC result of 1:125 dilution for M. luteus, 1:625 dilution for P. acnes, and 1:3125 dilution result for C. xerosis. The MLC results were effective at 1:5 dilution for M. luteus, 1:25 dilution for P. acnes, and 1:125 dilution for C. xerosis.

Accordingly a deodorant composition for suppressing M. luteus preferably includes dimethyl capramide in a concentration at least about 0.8 wt. %. Similarly, a deodorant composition for suppressing C. xerosis preferably includes dimethyl capramide in a concentration at least about 0.032 wt. %. An anti-acne composition for suppressing P. acnes preferably includes dimethyl capramide in a concentration at least about 0.16 wt. %.

### Example 2

A translucent deodorant stick was prepared from the formulation specified in Table 3 below, using dimethyl capramide instead of triclosan as an antimicrobial component.

TABLE 3
Anhydrous Deodorant Stick

| Item | Ingredient INCI Name (Trade Name) | wt. % |
|---|---|---|
| 1 | propylene glycol (Lyondell propylene glycol USP) | 18.1 |
| 2 | PPG-3 myristyl ether (Degussa Goldschmidt VARONIC) | 46.7 |
| | APM) | |
| 3 | cyclomethicone or cyclopentasiloxane (Rhodia MIRASIL | 28.2 |
| | CM5) | |
| 4 | sodium stearate (RTD*HallStar RTD OP-200) | 6.0 |
| 5 | dimethyl capramide (SPECTRASOLV DMDA) | 1.0 |

In an explosion-proof vessel with appropriate mixing and handling capabilities, items 1, 2, and 3 were added and mixing was started while heating to 80° C. Above 55° C., item 4 was added slowly (to prevent clumping). When the batch was crystal clear and completely free of undissolved item 4, item 5 was added, and very slow cooling with strong mixing without aeration (to prevent set up of the product on the vessel walls) was commenced. The product was filled to deodorant stick containers at a temperature between 58° C. and 52° C. (the product sets at about 50° C. At room temperature (about 23° C.) the product appears as a translucent, almost colorless, firm, deformation-resistant gel, and is virtually odorless. This formula yielded deodorant sticks with excellent application and wear properties.

### Example 3

A translucent deodorant stick was prepared from the formulation specified in Table 4 below, using dimethyl capramide instead of triclosan as an antimicrobial component.

TABLE 4
Hydroglycolic Deodorant Stick

| Item | Ingredient INCI Name (Trade Name) | wt. % |
|---|---|---|
| 1 | water [distilled, deionized] | 18.0 |
| 2 | propylene glycol (Lyondell propylene glycol USP) | 75.0 |
| 3 | sodium stearate (RTD*HallStar RTD OP-200) | 6.0 |
| 4 | dimethyl capramide (SPECTRASOLV DMDA) | 1.0 |

To a vessel with appropriate mixing and handling capabilities was added items 1 and 2, and mixing and heating to 80° C. were started. Above 70° C., item 3 was added slowly (to prevent clumping). When the batch was crystal clear and completely free of undissolved item 3, very slow cooling with strong mixing without aeration (to prevent set up of the product on the vessel walls) was commenced. At 70° C., item 4 was added. Mixing continued, and the product was filled to deodorant stick containers at between 68° C. and 62° C. (the product sets at about 60° C.). At room temperature (about 23° C.) the product appears as a translucent, almost colorless, firm, deformation-resistant gel, and is virtually odorless. This formula yielded deodorant sticks with excellent application and wear properties.

The foregoing description is given for clearness of understanding only, and no unnecessary limitations should be understood therefrom, as modifications within the scope of the invention may be apparent to those having ordinary skill in the art.

What is claimed is:

1. A composition comprising a bactericide comprising a dialkyl amide in a dermatologically acceptable carrier.

2. The composition of claim 1, wherein said dialkyl amide is derived from a carboxylic acid selected from C₈-C₁₈ monocarboxylic acids, and combinations thereof.

3. The composition of claim 2, wherein said dialkyl amide is a N,N-dimethyl amide.

4. The composition of claim 3, wherein said dialkyl amide is selected from the group consisting of N,N-dimethyldecanamide, N,N-dimethylundecanamide, N,N-dimethyllauramide, N,N-dimethyltridecanamide, N,N-dimethylmyristamide, N,N-dimethylmyristoleylamide, and combinations thereof.

5. The composition of claim 4, wherein said dialkyl amide comprises N,N-dimethyldecanamide.

6. The composition of claim 3, wherein said dialkyl amide is selected from the group consisting of N,N-dimethylcaprilamide, N,N-dimethylpelargonamide, N,N-dimethyldecanamide, N,N-dimethylundecanamide, N,N-dimethyllauramide, and combinations thereof.

7. The composition claim 1, in the form of a solution, lotion, cream, ointment, powder, suspension, stick, gel, aerosol, or nonaerosol pump spray.

8. The composition claim 1, in the form of a stick or gel.

9. The composition claim 1, wherein said carrier is anhydrous.

10. The composition claim 1, wherein said carrier comprises a vehicle selected from the group consisting of water, ethanol, dihydric alcohols, polyhydric alcohols, silicones, fatty alcohols, fatty acids, metallic soaps of fatty acids, fatty acid esters, and combinations thereof.

11. The composition claim 1, wherein said carrier comprises a dihydric and/or polyhydric alcohol-in-silicone emulsion.

12. The composition claim 1, wherein said bactericide is present in an amount of from 0.01 wt. % to 10 wt. %.

13. The composition claim 1, wherein said dialkyl amide is present in an amount of from 0.01 wt. % to 10 wt. %.

14. The composition claim 1, wherein said bactericide is present in an amount of from 0.1 wt. % to 2 wt. %.

15. The composition claim 1, wherein said dialkyl amide is present in an amount of from 0.1 wt. % to 2 wt. %.

16. The composition of claim 1, comprising a body odor suppressing effective amount of a bactericide comprising a dialkyl amide.

17. The composition of claim 16, wherein said dialkyl amide is present in an amount at least 0.032 wt. %.

18. The composition claim 16, further comprising an antiperspirant salt.

19. The composition claim 16, further comprising a fragrance material.

20. The composition of claim 1, comprising an acne suppressing effective amount of a bactericide comprising a dialkyl amide.

21. The composition of claim 20, wherein said dialkyl amide is present in an amount at least 0.16 wt. %.

22. The composition of claim 1, further comprising a germicide selected from the group consisting of triclosan, zinc phenol sulfonate, chlorhexidine digluconate, and combinations thereof.

23. A method of preparing a cosmetic product intended for human topical application, comprising the step of including in said product a composition a bactericide comprising a dialkyl amide.

24. A method for controlling a condition selected from the group consisting of acne, malodor associated with human perspiration, and combinations thereof, comprising the step of applying to the skin a composition comprising a bactericide comprising a dialkyl amide.
